(19) United States
(12) Patent Application Publication
(10) Pub. No.: US 2018/0244829 A1
(43) Pub. Date: Aug. 30, 2018

(54) THICKENING AGENT FOR AQUEOUS SYSTEMS, FORMULATIONS CONTAINING SAME AND USES

(71) Applicant: COATEX, Genay (FR)

(72) Inventors: Denis RUHLMANN, Genay (FR); Jean-Marc SUAU, Lucenay (FR); Yves MATTER, Quincieux (FR); Catherine CORFIAS ZUCCALLI, Villeurbanne (FR)

(73) Assignee: COATEX, Genay (FR)

(21) Appl. No.: 15/754,016

(22) PCT Filed: Sep. 29, 2016
(86) PCT No.: PCT/FR2016/052474

§ 371 Date: Feb. 21, 2018

(30) Foreign Application Priority Data
Oct. 5, 2015 (FR) 1559428

(51) Int. Cl. C08G 18/28; C08G 18/48; C08G 18/75; C08L 75/04; A61Q 19/00; A61K 8/87; C09D 7/43; C09D 11/03; C09D 11/102; C09D 175/04

(52) U.S. Cl. CPC C08G 18/282; C08G 18/4833; C08G 18/755; C08L 75/04; A61Q 19/00; A61K 8/87; A61K 2800/10; C09D 11/03; C09D 11/102; C09D 175/04; A61K 2800/48; C08L 2201/54; C09D 7/43

(57) ABSTRACT

The present invention relates to novel associative thickeners belonging to the HEUR (Hydrophobically modified Ethoxylated URethane) category and also to intermediate aqueous formulations containing such thickeners and to the final compositions, for example paint, lacquer, varnish or paper coating color compositions.

The present invention relates to novel associative thickeners belonging to the HEUR (Hydrophobically modified Ethoxylated Urethane) category. These products contain an associative compound comprising a carbon-based chain end and a methyl or ethyl branch. The present invention also relates to intermediate formulations containing such thickeners, and also to the use of these compounds as thickeners in final compositions, for example paint compositions.

Paints are made of fillers and pigments and at least one organic polymer called a binder. In addition to the fillers, pigments and binder, a paint composition also comprises at least one solvent (which is water in the case of water-phase paints), rheology additives, stability additives (storage, film formation, UV) and other additives for obtaining specific properties. The behavior and the properties of paints depend on the nature of their constituents, in particular of the binder, of the fillers and of the pigments, and also of the rheology additives. They generally contain one or more thickener(s), the function of which is to control the rheology of the compositions which contain it (them), both at the stage of their production and during their conveying, their storage or during their implementation. Given the diversity of the practical constraints at each of these steps, it is advantageous for the formulator to have a range of thickeners which exhibit different rheological behaviors when they are used in the final compositions. In addition, these thickeners can provide the compositions, for example the paints that contain them, with additional properties.

Among all the paint thickeners, the following are distinguished:
natural thickeners based on cellulose, also called cellulose ethers, of HEC type or of HMHEC (Hydrophobically Modified HEC) type,
acrylic thickeners of non-associative type, called ASE (Alkali Swellable Emulsion) and those of associative type, called HASE (Hydrophobically modified Alkali Swellable Emulsion) and
associative thickening polyurethanes of HEUR (Hydrophobically modified Ethoxylated URethane) type.

Thickening polyurethanes or HEURs result from the condensation between a compound of poly(alkylene glycol) type, a polyisocyanate and an associative compound of alkyl, aryl or aryalkyl type made of a hydrophobic end group.

Coatex is responsible for numerous research studies on paint thickeners. Moreover, Coatex sells the products of the Coapur® range, for example the Coapur® XS products, which are nonionic thickening polyurethanes providing rheological profiles that vary between the Newtonian type (high viscosity at high shear gradient) and/or the pseudoplastic type (high viscosity at low shear gradient).

Document EP 2664640 describes a water-soluble alkoxylated polymer which is nonionic. It is prepared by reacting a hydrophobic monovalent alcohol, a linear diol compound, a polyalkylene oxide and a diisocyanate. This polymer is intended to thicken cosmetic compositions and to make them transparent.

Document WO 2012 096125 also describes a water-soluble alkoxylated polymer. It is prepared by reacting a polyalkylene oxide, two monovalent hydrophobic alcohols and a diisocyanate. It is also intended to increase the viscosity of a cosmetic composition.

Document US 2005 187342 relates to thickening agents for aqueous dispersions which are based on an aqueous preparation of water-dispersible or water-soluble nonionic polyurethanes. They are obtained from a polyfunctional isocyanate, from a polyether polyol, from 2-(n-butyl)-1-octanol and from a polyol.

Document US 2008 108775 describes a thickening aqueous composition comprising a water-soluble or water-dispersible nonionic polyurethane. This polyurethane is obtained by reacting a hydrophilic polyol comprising ether or ester groups, a hydrophobic compound comprising at least one saturated or unsaturated, linear or branched hydrocarbon-based chain, and a diisocyanate.

The inventors have developed a novel thickening polyurethane which makes it possible to very notably increase the viscosity at high shear gradient and to thus confer on the composition which contains it a good dynamic behavior, that is to say a high viscosity at high shear gradient. This thickener can be categorized in the category of thickeners of Newtonian or “ICI builder” type.

A thickener of “ICI builder” type can be defined as a product which results in an increase in the ICI viscosity when its amount is increased in the paint composition, without significant increase in the viscosities at low shear rate (Brookfield viscosity).

This novel thickener can, for example, be used alone in a paint composition where it is required to have a high viscosity at high shear gradient (for example a silk or gloss paint containing a high binder content).

It can also be used in combination with a thickener of pseudoplastic type. Such a combination thus makes it possible to obtain a composition which exhibits good dynamic behavior associated with the presence of the Newtonian thickener and good static behavior associated with the presence of the thickener of pseudoplastic type.

Such a thickener can be formulated in aqueous phase and, because of its particular structure, it allows thickening of the final composition without requiring particular equipment or high shear energy.

This novel thickener has, at the chain ends, hydrophobic groups which result from the condensation of the alcohol of formula (I):

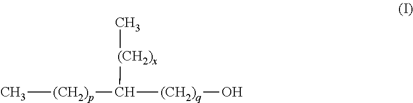

Document WO 2011/104599 (Coatex) describes rheology modifier polymers for aqueous systems, in particular paint compositions. The acrylic thickener-type thickeners described can contain, in particular, ethyl acrylate (EA), methacrylic acid (MAA) and polymerizable monomeric units of formula:

R-(AO)ₘ-(BO)ₙ—R′

in which:
R denotes a polymerizable unsaturated group, for example methacrylate,
A and B denote alkyl groups that are different than one another and have from 2 to 4 carbon atoms, for example ethylene oxide and propylene oxide,
m and n are integers less than 150, at least one of which is non-zero, and
R′ is made of at least one group of formula below:

CH₃—(CH₂)ₚ—CH(—CH₃)—(CH₂)_{q}—
in which:
   p and q denote integers, at least one of which is non-zero, and
   5<p+q<13.

Such acrylic polymers have a rheological profile different than that of the thickeners of the present invention. Namely, they fall into the category of acrylic thickeners of pseudoplastic type (high viscosity at low shear gradient).

### DEFINITIONS

The term “HEUR” is an acronym for “Hydrophobically modified Ethoxylated URethane”.

In the description of the present invention, unless otherwise indicated, the percentages expressed represent percentages by weight and are expressed relative to the total weight of the reference element. For example, when it is indicated that a polymer comprises 10% of a monomer or of a reagent, it is understood that the polymer comprises 10% by weight of this monomer or reagent relative to the total weight of this polymer.

In the description of the present invention, the expression “at least one” denotes one or more compound(s) (for example one or more alcohol compound(s), one or more poly(alkylene glycol(s)), one or more polyisocyanate(s)), such as a mixture of 2 to 5 compounds.

The term “formulation” is intended to mean the thickening intermediate entity comprising the polyurethane agent according to the invention formulated so as to be easier to use in the final composition to be thickened. For example, the thickening agent according to the invention can be formulated in the presence of water and of surface-active agents so as to be more easily pourable and easier to incorporate into the composition to be thickened at ambient temperature. The viscosity of the formulation before it is incorporated into the final aqueous composition is, for example, less than 10 000 mPa·s at 25° C. and at 100 revolutions per minute.

The term “composition” is intended to mean the final entity to be thickened or that has been thickened, comprising the polyurethane agent according to the invention, optionally formulated in the presence, for example, of water and of surface-active agents, and also all of the constituents thereof, the list of which depends on the final application. For example, the final composition comprises inorganic pigments and binders, if it is a paint composition.

### DETAILED DESCRIPTION OF THE INVENTION

The polyurethanes of the present invention are thickeners for aqueous compositions, for example aqueous paint compositions. They make it possible to obtain high viscosities at high shear gradient and to thus confer good dynamic behavior on the compositions. These thickening polyurethanes can be categorized in the category of thickeners of Newtonian or “ICI builder” type.

Some paint compositions, for example silk or gloss paints which contain few pigments (compared with a matt paint for example) and a lot of binder, must have the highest possible viscosity at high shear gradient. The term used is Cone Plan viscosity or ICI viscosity, denoted μ_{I} (mPa·s).

The thickening polyurethane of the present invention is entirely suitable for aqueous compositions of this type.

The thickening polyurethane of the present invention can also be used in combination with a thickener of pseudoplastic type. Such a combination thus makes it possible to obtain a composition which exhibits good dynamic behavior associated with the presence of the Newtonian thickener and good static behavior associated with the presence of the thickener of pseudoplastic type. It is thus possible to adjust the rheological behaviors of the aqueous formulations at low and high shear gradient.

This novel thickener has specific hydrophobic groups at the chain ends.

### HEUR Thickener:

A subject of the present invention relates to a thickener belonging to the HEUR (Hydrophobically modified Ethoxylated URethane) category. It is a nonionic associative thickening polymer for aqueous compositions.

The thickening polyurethanes or HEURs of the present invention result from the reaction between a reagent which confers the associativity and which is made of a hydrophobic end group, a compound of polyol type (for example poly(alkylene glycol)) and a polyisocyanate. In the context of the present invention, the terms “reaction”, “condensation” and “polycondensation” are used in an equivalent manner.

More specifically, it is a water-soluble thickening polyurethane resulting from the condensation:
a) of at least one alcohol of formula (I):

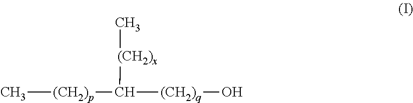

in which:
x represents 0 or 1,
p and q are integers, at least one of which is non-zero and
4<p+x+q<7,
b) of at least one poly(alkylene glycol) and
c) of at least one polyisocyanate.

It is these novel polyurethanes which make it possible, for example, to thicken a paint composition at high shear gradient (measurement of the ICI viscosity for example).

The polyurethane according to the present invention comprises, as constituent a), a compound of formula (I). Such a compound is, in the context of the present invention, known as an “associative compound comprising a carbon-based chain end and a methyl or ethyl branch”.

The expression “x represents 0 or 1” is intended to mean that the branch can be respectively methyl or ethyl.

Given formula (I), the expression “4<p+x+q<7” is intended to mean that the carbon-based chain comprises between 7 and 10 carbon atoms, for example 7, 8, 9 or 10 carbon atoms.

Thus, the compounds of formula (I) comprise:
a carbon-based chain end comprising from 7 to 10 carbon atoms, for example 8 carbon atoms or 9 carbon atoms and
a —CH₃ or —CH₂—CH₃ branch.

The water-soluble thickening polyurethane can result from the condensation of one or more alcohol(s) of formula (I).

According, to one embodiment of the present invention, the polyurethane is made up of several alcohols of formula (I). According to another embodiment, said polyurethane is made up of a single alcohol of formula (I).

According to one embodiment of the present invention, the alcohol a) has a formula (II):

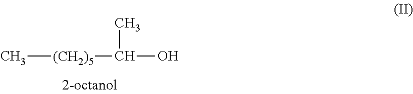

According to this embodiment, the compounds of formula (II) comprise:
a carbon-based chain end comprising 8 carbon atoms and
a —CH₃ branch (methyl branch).

According to another embodiment of the present invention, the alcohol a) has a formula (III):

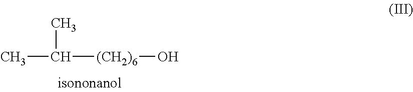

According to this embodiment, the compounds of formula (III) comprise:
a carbon-based chain end comprising 91 carbon atoms and
a —CH₃ branch (methyl branch).

Moreover, the polyurethane comprises, as constituent b), a poly(alkylene glycol).

The term “poly(alkylene glycol)” is intended to mean a polymer of an alkylene glycol derived from an olefin oxide. The poly(alkylene glycol) chains of the constituent b) according to the present invention contain a proportion of ethylene-oxy groups, a proportion of propylene-oxy groups and/or a proportion of butylene-oxy groups. The poly(alkylene glycol) chains according to the present invention can, for example, comprise a dominant proportion of ethylene-oxy groups in combination with a secondary proportion of propylene-oxy groups. Specific examples of alkylene glycol polymers comprise: poly(alkylene glycol)s having an average molecular weight of 1000 g/mol, 4000 g/mol, 6000 g/mol and 10 000 g/mol; polyethylene polypropylene glycols having a percentage of ethylene oxide of between 20% and 80% by weight and a percentage of propylene oxide of between 20% and 80% by weight.

According to one aspect of the present invention, the polyurethanes result from the condensation in particular of a poly(alkylene glycol) which is poly(ethylene glycol). it may for example be a polyethylene glycol) of which the molecular mass ranges between

2000 g/mol and 20 000 g/mol, for example between 8000 g/mol and 15 000 g/mol (limits included). By way of example, mention may be made of poly(ethylene glycol) (or PEG) having a molecular mass ranging between 10 000 g/mol and 12 000 g/mol (limits included).

Moreover, the polyurethane comprises, as constituent c), a polyisocyanate.

The term “polyisocyanate” is intended to mean a compound which comprises at least 2 isocyanate functional groups —N═C═O.

According to one aspect of the present invention, the polyurethanes result from the condensation in particular of a polyisocyanate which is chosen from the group consisting of toluene diisocyanate, toluene diisocyanate dimers, toluene diisocyanate trimers, 1,4-butane diisocyanate, 1,6-hexane diisocyanate, isophorone diisocyanate, 1,3-cyclohexane diisocyanate, 1,4-cyclohexane diisocyanate, 4,4′diisocyanatodicyclohexylmethane, 1-methyl-2,4-diisocyanatocyclohexane, diphenyl methylene diisocyanate (MDI), for example 2,2′-MDI, 2,4′-MDI, 4n4′-MDI or mixtures thereof, dibenzyl diisocyanate, a mixture of 1-methyl-2,4-diisocyanatocyclohexane and of 1-methyl-2,6-diisocyanatocyclohexane, hexamethylene diisocyanate biuret, hexamethylene diisocyanate biuret dimers, hexamethylene diisocyanate biuret trimers, and a mixture of at least two of these compounds.

According to one aspect of the invention, said polyurethane results from the condensation of:
a) 1% to 29% by weight of at least one compound of formula (I), (II) and/or (III),
b) 70% to 98% by weight of at least one poly(alkylene glycol) and
c) 1% to 29% by weight of at least one polyisocyanate,
   the sum of these mass percentages being equal to 100%.

According to another aspect of the invention, said polyurethane results from the condensation of:
a) 3% to 10% by weight of at least one compound of formula (I), (II) and/or (III),
b) 80% to 94% by weight of at least one poly(alkylene glycol) and
c) 3% to 10% by weight of at least one polyisocyanate,
   the sum of these mass percentages being equal to 100%.

According to one embodiment, the present invention relates to a water-soluble thickening polyurethane resulting exclusively from the condensation of the following 3 constituents:
a) an alcohol of formula (I):

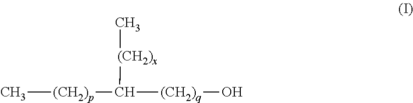

in which:
x represents 0 or 1,
p and q are integers, at least one of which is non-zero, and
4<p+x+q<7,
b) a poly(alkylene glycol) and
c) a polyisocyanate.

The production of polyurethanes, which belong to the family of thickeners of HEUR type, is known to those skilled in the art who will be able to refer to the teaching of the documents mentioned above in the technological background of the present invention.

Another subject of the present invention also relates to a method for preparing a polyurethane as described above, said method consisting of a condensation of its various constituents.

### Formulation of the HEUR Thickener:

The polyurethane according to the invention can be formulated or co-formulated with other constituents or components.

Thus, the present invention also relates to an aqueous formulation comprising a polyurethane according to the invention, as described above.

This thickening aqueous formulation is intended to be incorporated into a final composition, for example a paint, a paper coating color or a detergent composition.

The polyurethane according to the invention can be co-formulated in the presence of water.

According to one embodiment, said aqueous formulation according to the invention consists of:
1) 5% to 50% by weight of at least one polyurethane according to the invention, as described above and
2) 50% to 95% by weight of water,
   the sum of these mass percentages being equal to 100%.

According to another embodiment, said aqueous formulation according to the invention consists of:
1) 5% to 25% by weight of at least one polyurethane according to the invention, as described above and
2) 75% to 95% by weight of water,
   the sum of these mass percentages being equal to 100%.

The polyurethane according to the invention can be co-formulated in water, in the presence of at least one surface-active agent. This surface-active agent makes it possible to formulate the thickener in the form of a less viscous liquid aqueous solution which can thus be more easily used by the formulator.

Thus, according to one embodiment of the present invention, said aqueous formulation comprises a polyurethane, as described above, and also water and at least one surface-active agent.

The term “surfactant” or “surface-active agent” is intended to mean a molecule or a polymer made up of at least one hydrophilic part and of at least one hydrophobic part.

The surface-active agent used in the context of the present invention can be of different nature, for example it can be anionic or nonionic.

This surface-active agent can be selected from the classes of ionic surface-active agents (in this case preferably anionic) and/or nonionic surface-active agents and/or mixed surface-active agents (comprising a nonionic and anionic structure in the same molecule). The preferred surface-active agent is composed of at least one surface-active agent selected from the class of nonionic surface-active agents, optionally in the presence of an anionic surface-active agent.

Among the suitable anionic surface-active agents, mention may be made of sodium, lithium potassium, ammonium or magnesium salts derived from alkyl ether sulfates with alkyl(s) ranging from C₆ to C₁₂, in linear, iso, oxo, geminal, cyclic or aromatic configuration, or C₁₂ alkyl sulfates, alkyl phosphate esters or dialkyl sulfosuccinates. The anionic surface-active agents are preferably used with at least one nonionic surface-active agent.

As examples of mixed surface-active agents, mention may be made of alkoxylated alkyl, phenol sulfonates. The nonionic surface-active agents can be used alone or in combination with an anionic surface-active agent. As preferred examples of suitable nonionic surface-active agents, mention may be made of: ethoxylated C₄-C₁₈ alcohols (2 to 15 OE), ethoxylated C₄-C₁₈ Guerbet alcohols (2 to 40 OE), ethoxylated C₁₀-C₁₈ monobranched alcohols (2 to 40 OE), C₁₈ sorbitol esters, ethoxylated sorbitol esters (2 to 20 OE units), ethoxylated C₄-C₁₈ acids (less than 15 OE), ethoxylated castor oil (30 to 40 OE), ethoxylated hydrogenated castor oil (7 to 60 OE), esters such as glycerol palmitate, glycerol stearate, ethylene glycol stearate, diethylene glycol stearate, propylene glycol stearate, polyethylene glycol stearate 200 and ethoxylated C₁₈ esters (2 to 15 OE). The hydrophobic chains can correspond to linear, iso, oxo, cyclic or aromatic structures.

According to one embodiment, the formulation comprises at least one nonionic surface-active agent optionally combined with at least one anionic surface-active agent, at a total content by weight ranging from 0.1% to 40% by weight, for example from 5% to 20% by weight or from 10% to 17% by weight. In this case, the weight ratio between the two surface-active agents can, for example, range between 25/75 and 75/25.

According to one embodiment of the present invention, the polyurethane of the present invention is formulated in the presence of more than two surface-active agents, for example three or four.

According to one embodiment, said aqueous formulation according to the invention consists of:
1) 2% to 50% by weight of at least one polyurethane according to the invention, as described above, preferably 5% to 30% by weight,
2) 0.1% to 40% by weight of at least one surface-active agent, preferably 2% to 30% by weight, and
3) 10% to 93% by weight of water, preferably 40% to 85% by weight,
   the sum of these mass percentages being equal to 100%.

The polyurethane according to the invention can be formulated in a water-miscible solvent. The main reason for the addition of an organic co-solvent is to lower the viscosity of this polyurethane in water, in order to facilitate the handling thereof. The polyurethane is, for example, formulated with one or more polar solvent(s) belonging in particular to the group made up of water, methanol, ethanol, propanol, isopropanol, butanols, acetone, tetrahydrofuran or mixtures thereof.

Two particular examples of water-miscible organic solvents are:
diethylene glycol monobutyl ether (also known as Butyl Carbitol™) or ethylene glycol ether or propylene glycol ether and
butylene glycol ether.

The viscosity of the polyurethane as it is, before it is incorporated into a paint composition for example, is advantageously less than 10 000 mPa·s at 25° C. and at 100 revolutions per minute, such that it is easier to pour from the storage container and is more rapidly incorporated into the composition to be thickened at ambient temperature. The water-miscible solvent chosen for such commercial compositions has, up until now, been exclusively an organic solvent.

The polyurethane according to the invention can be co-formulated in water, in the presence of a coalescent agent. In a manner equivalent to a solvent, the coalescent agent makes it possible to formulate the thickener in the form of a less viscous liquid aqueous solution which can thus be more easily used by the formulator.

According to one embodiment, said aqueous formulation according to the invention consists of:
1) 5% to 50% by weight of at least one polyurethane according to the invention, as described above,
2) 5% to 30% by weight of at least one solvent and/or coalescent agent and
3) 20% to 75% by weight of water,
   the sum of these mass percentages being equal to 100%.

According to one aspect of the invention, the aqueous formulation also comprises at least one additive selected from the group consisting of a biocide, a solvent, an anti-foaming agent, a pH regulator, a coalescent agent, an encapsulating agent and mixtures thereof.

The term “biocide” is intended to mean a chemical substance intended to destroy, repel or render harmless pest organisms, to prevent the action thereof or to combat them in any other ways, by a chemical or biological action.

The term “anti-foaming agent” is intended to mean a substance or a formulation intended to destroy the air bubbles in a homogeneous or heterogeneous liquid medium (or at its surface) or to prevent the formation thereof.

The term “pH regulator” or “pH-regulating agent” is intended to mean a chemical compound which makes it possible to adjust the pH to the expected value. For example, the pH-regulating agent can increase the pH; this is the case with the bases such as NaOH. Alternatively, the pH-regulating agent can decrease the pH; this is the case with acids.

The term “coalescent agent” is intended to mean an agent used in paints, which makes it possible to lower the Minimum Film Formation Temperature (MFFT) of paint to a temperature suitable for the desired application conditions (for example, an MFFT of 5° C. for an outside application). By way of examples of coalescent agents according to the invention, mention may be made of propylene glycol, butyl glycol, 2,2,4-trimethyl-1,3-pentanediol monoisobutyrate or 2,2,4-trimethyl-1,3-pentanediol diisobutyrate, 2,2,4-trimethyl-1,3-pentanediol monoisobutyrate, and glycol ether derivatives of Dowanol® type.

The term “encapsulating agent” is intended to mean an agent that creates a hydrophobic environment, for example a solvation cage. As encapsulating agent, mention is in particular made of cyclodextrin.

According to one embodiment, said aqueous formulation according to the invention consists of:
1) 2% to 50% by weight of at least one polyurethane according to the invention, as described above, preferably 2% to 30% by weight,
2) 0.1% to 40% by weight of at least one surface-active agent, preferably 5% to 30% by weight,
3) 10% to 93% by weight of water, preferably 40% to 85% by weight, and
4) 0% to 5% by weight of at least one other additive chosen from the group consisting of a biocide, a solvent, an anti-foaming agent, a pH regulator, a coalescent agent, an encapsulating agent and mixtures thereof, preferably 0.5% to 4% by weight,
   the sum of these mass percentages being equal to 100%.

### Final Aqueous Composition and Uses of the Polyurethane:

A subject of the present invention consists of an aqueous composition comprising a polyurethane according to the invention or a thickening aqueous formulation according to the invention, said final aqueous composition being selected from the group consisting of a paint, a putty, a thick coating, a waterproof coating, a lacquer, a varnish, an ink, a slurry, a paper coating color, a cosmetic composition and a detergent composition.

Said composition is thickened by means of a polyurethane or of a thickening aqueous formulation according to the invention.

Moreover, the present invention also relates to the use of a polyurethane according to the invention or of an aqueous formulation according to the invention, for thickening an aqueous composition, said composition being selected from the group consisting of a lacquer, a varnish, a paint, a putty, a thick coating, a waterproof coating, an ink, a slurry, a paper coating color, a cosmetic composition and a detergent composition.

More specifically, according to one embodiment, the present invention, relates to the use of a polyurethane according to the invention or of an aqueous formulation according to the invention, for increasing the viscosity of the aqueous composition at high shear gradient.

The expression “increasing the viscosity of the aqueous composition at high shear gradient” is intended to mean that the viscosity at high shear gradient of the aqueous composition containing a given amount of polyurethane is greater than that of the same aqueous composition containing a lower amount of the same polyurethane. In other words, increasing the amount of polyurethane in the aqueous composition results in increasing the viscosity at high shear gradient of this composition.

According to one embodiment, the use of a polyurethane according to the invention or of an aqueous formulation according to the invention makes it possible to selectively increase the viscosity of the aqueous composition at high shear gradient (for example, ICI viscosity), that is to say without significant increase in the viscosities at low and medium shear rate (for example, Brookfield and Stormer viscosities). In other words, increasing the amount of polyurethane in the aqueous composition results in increasing the viscosity at high shear gradient of this composition, without this increase in amount resulting in significantly increasing the viscosities at low and medium shear gradient.

The inventors have developed a novel thickening polyurethane which makes it possible to very notably increase the viscosity at high shear gradient and to thus confer on the composition which contains it a good dynamic behavior, that is to say a high viscosity at high shear gradient. This thickener can be categorized in the category of thickeners of Newtonian or “ICI builder” type.

A thickener of “ICI builder” type can be defined as a product which results in an increase in the ICI viscosity when its amount is increased in the paint composition, without significant increase in the viscosities at low and medium shear rate (Brookfield and Stormer viscosities).

This novel thickener can, for example, be used alone in a paint composition where it is required to have a high viscosity at high shear gradient (for example a silk or gloss paint containing a high binder content).

According to one embodiment, the aqueous composition to be thickened is of the type of a gloss paint, a semi-gloss paint, a silk paint or any other paints with a low Pigment Volume Concentration (PVC).

The “pigment volume concentration” is defined by the following formula:

PVC (%)=100×V_{c}/(V_{c}+V₁)
with V_{c} representing the volume of the mineral fillers and
V₁ representing the volume of binders in the paint formulation.

According to another embodiment, the aqueous composition to be thickened is of the type of a paint with a medium or high Pigment Volume Concentration (PVC), which varies between egg-shell paint and matt paint. In this case, the thickening polyurethane of the present invention can be combined with another thickener which has a pseudoplastic profile.

According to one embodiment of the present invention, said polyurethane or said aqueous polyurethane formulation is used as a leveling agent for said aqueous composition. The polyurethane according to the invention makes it possible, for example, to increase the leveling value of the paint which contains it, that is to say the self-smoothing capacity of the paint during application. This value can, for example, be measured on a contrast chart Leneta, standard ASTM D4062, called “flow and leveling”.

According to one embodiment of the present invention, the use of said polyurethane or of said aqueous formulation of polyurethane in an aqueous paint composition makes it possible in particular to improve the following applicative properties:
brush drag,
film build,
hiding power due to a more even deposition, and
spatter resistance.

According to one aspect of the present invention, the aqueous composition comprises from
0.02% to 5% by weight of active ingredient of said thickener.

According to another aspect of the present invention, the aqueous formulation comprises from 0.05% to 2% by weight of active ingredient of said thickener.

The term “weight of active ingredient” is intended to mean the dry weight of polyurethane according to the invention, independently of the co-formulation ingredients.

According to yet another aspect of the present invention, the aqueous composition comprises at least one mineral filler selected from the group consisting of calcium carbonate, kaolin, talc and silicate and/or at least one pigment selected from the group consisting of titanium dioxide, iron oxide and zinc.

According to one aspect of the invention, the aqueous composition is a paint and comprises at least one dispersing agent, at least one mineral filler or one mineral pigment, at least one binder, at least one biocide, at least one anti-foaming agent and optionally one surface-active agent, a surface agent and/or a coalescent agent, a solvent.

The examples which follow make it possible to understand the present invention more clearly, without limiting the scope thereof.

### EXAMPLES

The viscosity of the test formulations or of the paint compositions is determined at various shear rates:
at low shear rate: the Brookfield viscosity (Bk) which is measured using a Brookfield RVT-type viscometer, in the unstirred flask, at a temperature of 25° C. and at two rotation speeds of 10 and 100 revolutions per minute with the appropriate spindle.

The reading is performed after 1 minute of rotation. Two Brookfield viscosity measurements, respectively denoted μ_{Bk10} and μ_{Bk100} (mPa·s), are thus obtained,
at medium shear rate: the Stormer viscosity, denoted μ_{S} (Krebs Units or KU) and
at high shear rate: the Cone Plan viscosity or ICI viscosity, denoted μ_{I} (Poise or P).

### Example 1

This example illustrates the use of a thickener according to the invention in an aqueous silk paint formulation, without solvent, the make-up of which is given in table 1 below. It illustrates the thickening capacity of a polyurethane according to the invention (tests 1-3 and 2-3), using a compound of formula (III). In parallel, this example also illustrates polyurethanes outside the invention (tests 1-1 and 2-1) and a HASE acrylic thickener according to (tests 1-2 and 2-2).

### Tests 1-1 and 2-1 (Outside the Invention):

Said polyurethane results from the condensation of, expressed as percentage by weight relative to the total weight of the polyurethane:
2.7% by weight of a non-ethoxylated linear alcohol of formula CH₃—(CH₂)₇—OH,
92.8% by weight of PEG 10 000 and
4.5% by weight of isophorone diisocyanate (IPDI).

### Tests 1-2 and 2-2 (According to the Invention):

Said polyurethane results from the condensation of, expressed as percentage by weight relative to the total weight of the polyurethane:
3.5% by weight of a compound of formula (II):

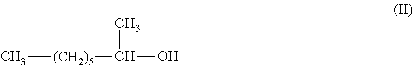

90.5% by weight of PEG 10 000 and
6.0% by weight of isophorone diisocyanate (IPDI).

### Tests 1-3 and 2-3 (According to the Invention):

Said polyurethane results from the condensation of, expressed as percentage by weight relative to the total weight of the polyurethane:
3.4% by weight of a compound of formula (III):

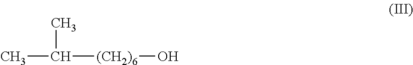

91.3% by weight of PEG 10 000 and
5.3% by weight of isophorone diisocyanate (IPDI).

The polyurethanes are formulated in water in the presence of a surface-active agent which is a C8-C10 fraction of an alkoxylated fatty alcohol (Simulsol®OX1008). The PU/surfactant/water ratios are 20/5/75.

All the results have been grouped together in tables 2 and 3 below.

For each of the tests, the μ_{Bk10}, μ_{Bk100}, μ_{I} (in P) and μ_{S} (in Krebs Units measured with the standard module) viscosities were determined, according to the methods described above at T=0 and at T=24 h, at ambient temperature.

TABLE 1
Paint constituent Mass (g)

| | |
|---|---|
| Water | 99.45 |
| Dispersant (Coadis ® BR3, Coatex) | 3.9 |
| Biocide (Acticide ® MBS, Thor) | 1.3 |
| Anti-foaming agent (Airex ® 901W, Tego) | 1.31 |
| NH₄OH (28%) | 0.5 |
| TiO₂ (RHD2 ® Hunstman) | 122.2 |
| CaCO₃ (Omyacoat ® 850OG, Omya) | 84.5 |
| Binder (Acronal 290D, BASF) | 270.6 |
| Monopropylene glycol | 6.5 |
| Texanol ® (Eastman) | 6.5 |
| Anti-foaming agent (Tego ® 825, Tego) | 0.65 |
| PU thickener (according to the tests) | Series 1: 28.6 |
| | Series 2: variable |
| | (see table 3) |
| Water | q.s.f. 650 g in total |

TABLE 2

| | | Test 1-1 | Test 1-2 | Test 1-3 |
|---|---|---|---|---|
| | | OINV | INV | INV |
| Amount | | 0.88 | | |
| (% by weight/ | | | | |
| total formula) | | | | |
| T = 0 | μ_{Bk10} | 2 660 | 1 730 | 1 795 |
| | μ_{Bk100} | 1 584 | 957 | 1 110 |
| | μ_{S} | 95 | 81 | 85 |
| | μ_{I} | 2.5 | 2.2 | 2.0 |
| T = 24 h | μ_{Bk10} | 3 320 | 1 730 | 2 110 |
| | μ_{Bk100} | 2 018 | 1 012 | 1 333 |
| | μ_{S} | 102 | 84 | 91 |
| | μ_{I} | 2.5 | 2.2 | 2.0 |

TABLE 3

| | | Test 2-1 | Test 2-2 | Test 2-3 |
|---|---|---|---|---|
| | | OINV | INV | INV |
| Amount | | 1.44 | 1.44 | 1.44 |
| (% by weight/ | | | | |
| total formula) | | | | |
| T = 0 | μ_{Bk10} | 4 560 | 2 560 | 2 560 |
| | μ_{Bk100} | 2 580 | 1 360 | 1 492 |
| | μ_{S} | 118 | 90 | 97 |
| | μ_{I} | 3.9 | 3.9 | 3 |
| T = 24 h | μ_{Bk10} | 5 800 | 3 240 | 3 240 |
| | μ_{Bk100} | 3 492 | 1 872 | 2 108 |
| | μ_{S} | 126 | 99 | 109 |
| | μ_{I} | 4 | 4 | 3.2 |

OINV: Outside the INVention
INV: According to the INVention

The polyurethanes according to the present invention (tests 1-2, 2-2, tests 1-3 and 2-3) exhibit a Newtonian-type rheological profile: low viscosity at low and medium shear gradient.

By comparison of the results presented for tests 1-3 and 2-3 (according to the invention), a significantly improved thickening at high shear rate (μ_{I}) is noted in the paint formulation, whereas the Brookfield and Stormer viscosities change more moderately; this is characteristic of a Newtonian thickener of “ICI builder” type, which allows a selective increase in the ICI viscosity as a function of the amount.

The polyurethane of the present invention thus offers a good compromise between Newtonian behavior (which makes it possible to obtain low viscosities at low shear gradient and medium shear gradient) and “ICI builder” characteristic which allows a selective increase in the ICI viscosity as a function of the amount used. The formulator can, thus, adjust the amount of thickener as a function of the rheological behavior desired at high shear gradient.

The polyurethane outside the invention of test 1-1 makes it possible to obtain a thickening at high shear rate (μ_{I}) of the same order of magnitude as that obtained with the polyurethanes according to the invention of tests 1-2 and 1-3. Nevertheless, it is noted that the Brookfield and Stormer viscosities obtained with the polyurethane outside the invention of test 1-1 are overall higher than those obtained with the polyurethane according to the invention of tests 1-2 and 1-3 at an identical amount (0.88% by weight relative to the total weight of the composition).

The polyurethane of tests 1-1 and 2-1 generates an ICI viscosity that can be modulated as a function of the amount added to the formula, but it is coupled with viscosities that are too high at low and medium shear rates. The profile of this polyurethane is not sufficiently Newtonian.

1. A water-soluble thickening polyurethane, obtained from a process comprising: condensing
a) at least one alcohol of formula (I): 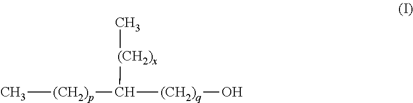
in which:
x represents 0 or 1,
p and q are integers, at least one of which is not zero, and
4<p+x+q<7;
b) at least one poly(alkylene glycol); and
c) at least one polyisocyanate.

2. The polyurethane according to claim 1, wherein the alcohol a) has a formula (II):

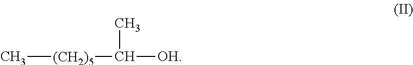

3. The polyurethane according to claim 1, wherein the alcohol a) has a formula (III):

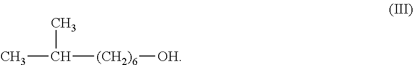

4. The polyurethane according to claim 1, obtained from a process comprising:
reacting
1% to 29% by weight of the at least one alcohol a),
70% to 98% by weight of the at least one poly(alkylene glycol) b), and
1% to 29% by weight of the at least one polyisocyanate c),
wherein a sum of the weight percentages of a), b), and c) is equal to 100%.

5. The polyurethane according to claim 1, wherein the poly(alkylene glycol) b) is a polyethylene glycol) whose molecular mass ranges between 2000 g/mol and 20 000 g/mol.

6. An aqueous formulation, comprising: the polyurethane according to claim 1.

7. The aqueous formulation according to claim 6, further comprising: water and at least one surface-active agent.

8. The aqueous formulation according to claim 6, further comprising: at least one additive selected from the group consisting of a biocide, a solvent, an anti-foaming agent, a pH regulator, a coalescent agent, and an encapsulating agent.

9. The aqueous formulation according to claim 6, consisting of:
2% to 50% by weight of at least one polyurethane,
0.1% to 40% by weight of at least one surface-active agent,
10% to 93% by weight of water, and
0% to 5% by weight of at least one other additive selected from the group consisting of a biocide, a solvent, an anti-foaming agent, a pH regulator, a coalescent agent, and an encapsulating agent,
wherein a sum of the weight percentages is equal to 100%.

10. A method for thickening an aqueous composition, the method comprising: introducing the polyurethane according to claim 1 into the composition, which is selected from the group consisting of a lacquer, a varnish, a paint, a putty, a thick coating, a waterproof coating, an ink, a slurry, a paper coating color, a cosmetic composition, and a detergent composition.

11. The method according to claim 10, wherein the polyurethane is a leveling agent for the aqueous composition.
